(19) United States
(12) Patent Application Publication
(10) Pub. No.: US 2006/0057126 A1
(43) Pub. Date: Mar. 16, 2006

(54) Device and method for hair growth from stem cells

(76) Inventor: Nikolai Tankovich, San Diego, CA (US)

Correspondence Address: JOHN R. ROSS, P.O. BOX 2138, DEL MAR, CA, 92014, US

(21) Appl. No.: 11/229,089

(22) Filed: Sep. 16, 2005

Related U.S. Application Data
(60) Provisional application No. 60/610,220, filed on Sep. 16, 2004.

(51) Int. Cl. A61K 35/36

(52) U.S. Cl. 424/93.7

(57) ABSTRACT

A method for utilizing an individual's undifferentiated papilla and/or bulge area stem cells to stimulate hair growth. The inventor has discovered that bulge area stem cells can be harvested, isolated, cloned, and successfully transplanted into an area of the donor's skin where increased growth of hair is desired to increase hair growth therein. In the first step of the method, a donor section of skin is identified having growth of the type of hair for which increased growth at the recipient site is sought. Since hair types differ according to their anatomical site, it is generally desirable to match the hair produced by the donor stem cells to the type of hair that is desired at the recipient site. For example, in treatment of male pattern baldness, tissue samples are harvested from an area of the scalp that still exhibits vigorous growth. Once the donor site is identified, it is anesthetized locally using any convenient means and a plurality of tissue samples are obtained from the donor site. The tissue samples preferably contain hair follicles with intact undifferentiated papilla and/or dermal stem cells, as well as immediately surrounding tissues. Any method of tissue sampling can be employed, for example, punch biopsy, so long as viable stem cells can be obtained. The stem cells are cloned to multiply the number of cells preferably by about 10 to 1,000 times or more. Some of the stem cells can be frozen for later use. Some of the stem cells are inserted into skin regions where hair restoration is desired. Various techniques are disclosed for inserting the stem cells into the skin.

This Application claims the benefit of Provisional Patent Application, Ser. No. 60/610,220 filed Sep. 16, 2004. The present invention relates to stem cells and to processes for promoting hair growth.

### BACKGROUND OF THE INVENTION

Millions of American men are going bald and they do not like it. Some give up and shave their heads of what little hair remains. Others try to comb it in a way that hides the hair loss. Some wear toupees or wigs. Millions of dollars are spent in the United States for hair restorations techniques that are mostly ineffective. Hair loss is also a problem for many women.

It is known that papilla and mid-derm bulge area stem cells play an important role in the hair growth cycle. Several groups of researchers have reported on the key role in regulation of hair growth found in bulge area stem cells.

What is needed is a technique for growing hair that works.

### SUMMARY OF THE INVENTION

The present invention provides a method for utilizing an individual's undifferentiated papilla and/or bulge area stem cells to stimulate hair growth. The inventor has discovered that bulge area stem cells can be harvested, isolated, cloned, and successfully transplanted into an area of the donor's skin where increased growth of hair is desired to increase hair growth therein. In the first step of the method, a donor section of skin is identified having growth of the type of hair for which increased growth at the recipient site is sought. Since hair types differ according to their anatomical site, it is generally desirable to match the hair produced by the donor stem cells to the type of hair that is desired at the recipient site. For example, in treatment of male pattern baldness, tissue samples are harvested from an area of the scalp that still exhibits vigorous growth. Once the donor site is identified, it is anesthetized locally using any convenient means and a plurality of tissue samples are obtained from the donor site. The tissue samples preferably contain hair follicles with intact undifferentiated papilla and/or dermal stem cells, as well as immediately surrounding tissues. Any method of tissue sampling can be employed, for example, punch biopsy, so long as viable stem cells can be obtained. The stem cells are cloned to multiply the number of cells preferably by about 10 to 1,000 times or more. Some of the stem cells can be frozen for later use. Some of the stem cells are inserted into skin regions where hair restoration is desired. Various techniques are disclosed for inserting the stem cells into the skin.

### BRIEF DESCRIPTION OF THE DRAWINGS

FIG. 1 is a cross-sectional representation of human skin.

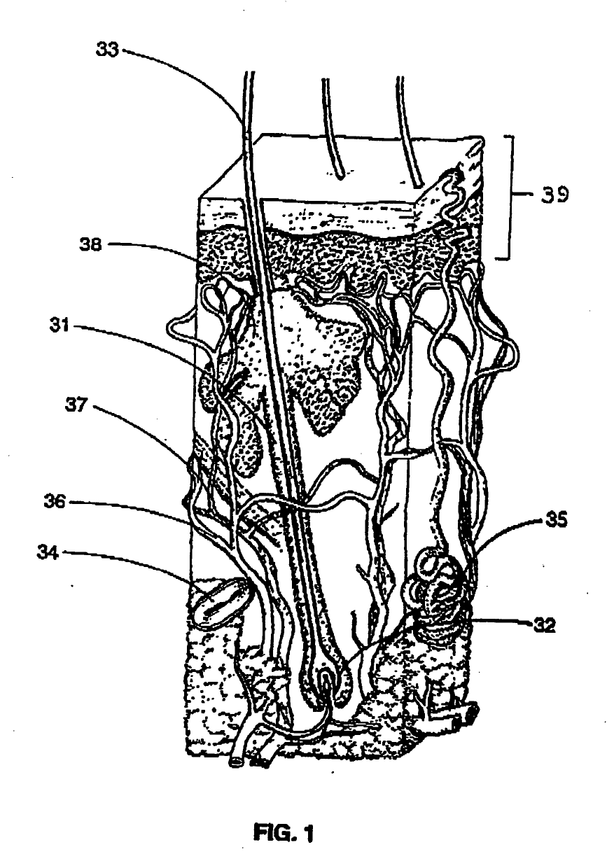

FIGS. 2A AND 2B show a process of hair waxing in the liquid medium containing stem cells or hair growth stimulating composition and is a subject to be delivered.

FIG. 3 shows a process of pulling hair in the medium with stem cells or hair growth stimulating composition.

FIG. 4 shows a device for delivering stem cells to hair ducts.

FIG. 5 shows a device for monitoring glucose in hair ducts.

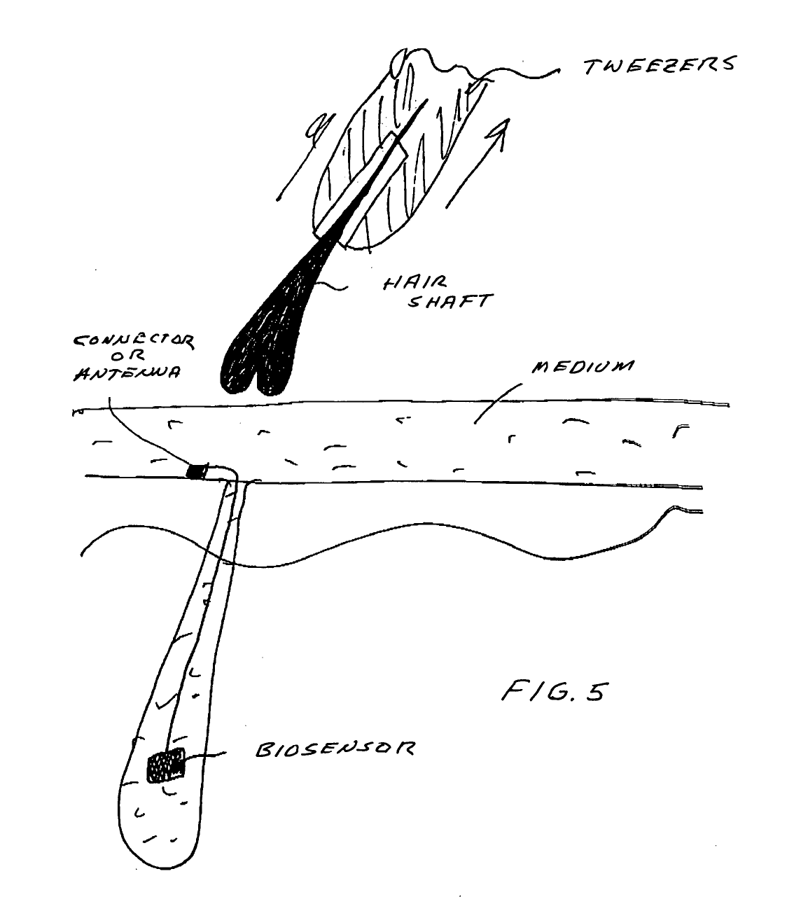

### DETAILED DESCRIPTION OF PREFERRED EMBODIMENTS

### Collecting Stem Cells

Undifferentiated stem cells are separated out from the mid-derm bulge area of hair papilla in the tissue samples. For example, the tissue samples can be micro-surgically dissected to locate and separate out the stem cells. Cells may also be collected from tissue other than hair type tissue. For example, stem cells from fat tissue may be collected. However, as explained in the next section. Transfection of stem cells into hair cells is more difficult than transfection of stem cells from the mid-derm area of the papilla since these latter stem cells are already partially differentiated into the direction of skin, nail and hair tissue. Stem cells used in preferred processes may be those taken form the hair growth patient being treated but they may also be stem cells from other people or previously frozen stem cells from a storage location.

### Cloning and Transfecting the Cells

Cloning

The separated stem cells are then preferably cloned by culturing them in an appropriate growth medium, such as Dulbecco's modified Eagle's medium (DMEM) with fetal calf serum, for a sufficient time to allow proliferation and differentiation of the cells. Generally, the cells are cloned to a cell density of about 40 cells per cubic centimeter. A single growth cycle will require approximately 21 to 28 days. During culture, the medium is kept at about body temperature (37° C.). Persons skilled in the art will understand that any one of a number of alternative growth media can be used to foster proliferation and differentiation of the stem cells. Once the desired cell density is achieved, for instance after about 2 to 3 passages, the cloned cells can be examined microscopically to detect the vital cells. Healthy differentiated stem cells are generally identified by applying a vital dye, such as Hoehst 33258 or Hoehst 33342 fluorescent dyes, incubating the cells for about 30 minutes, and then determining which of the cells fluoresce. Cells can be multiplied by factors such as 10, 1,000, or 1,000,000 or more.

Transfecting

While being cultured, it is important to assure that the cells are properly differentiated into hair cells. Techniques to assure this proper differentiation will depend on the cells used to start the process. Materials are available to promote this differentiation. These materials include cytokines, such as IL-1, Il-6 and Il-8; growth factors such as TFG and genetic materials such as vectors, plasmids and promoters.

### The Carrier Solution

A sterile suspension of the cells in a biologically acceptable carrier medium, such as normal saline, is then prepared for inoculation or transplant into one or more recipient sites of the same individual from which the stem cells were harvested. Suitable carrier media include aqueous or non-aqueous solutions, suspensions, and emulsions. Examples of non-aqueous solutions are propylene glycol, polyethylene glycol, and injectable organic esters, such as ethyl oleate. Aqueous carriers include water, alcoholic-aqueous solutions, and suspensions, including saline and buffered media.

### Applying the Stem Cells to the Skin Region

Stem cells can be applied to skin regions for hair restoration by a variety of techniques. Some of these techniques are described below.

Grafting

For inter-dermal grafting, the suspension of differentiated stem cells should be at a density of about 3 to about 10 percent by volume. For grafting of the differentiated stem cells, the recipient site is prepared by scraping the skin surface and making superficial incisions of about 200 microns in depth. The solution of stem cells is delivered to the recipient site, generally by pipette, and the site is covered with a sterile bandage, such as Tegaderm™.

Insertion into Hair Ducts

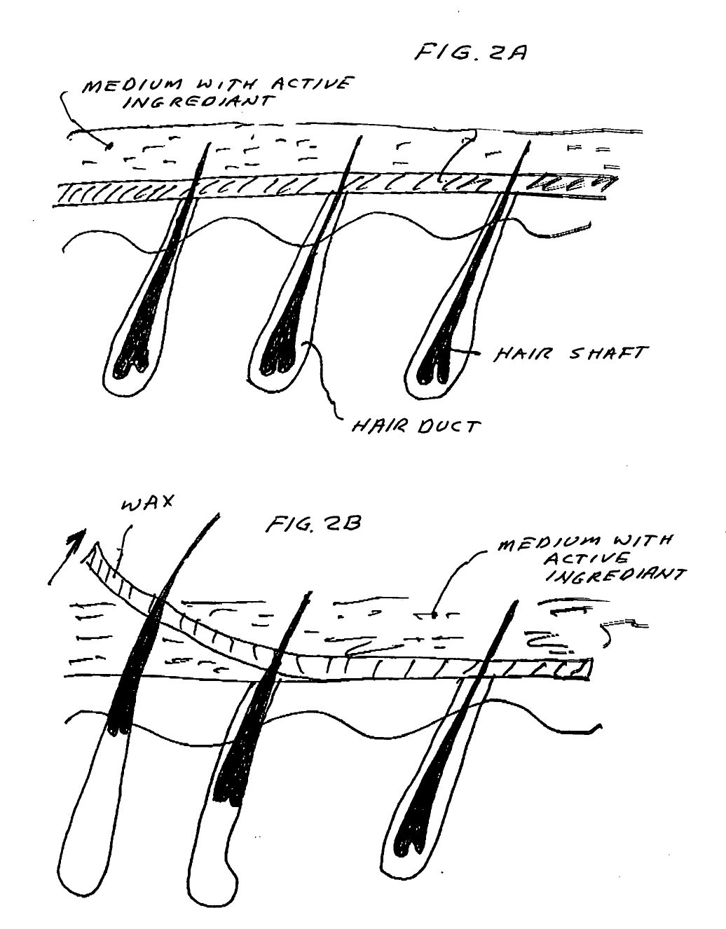

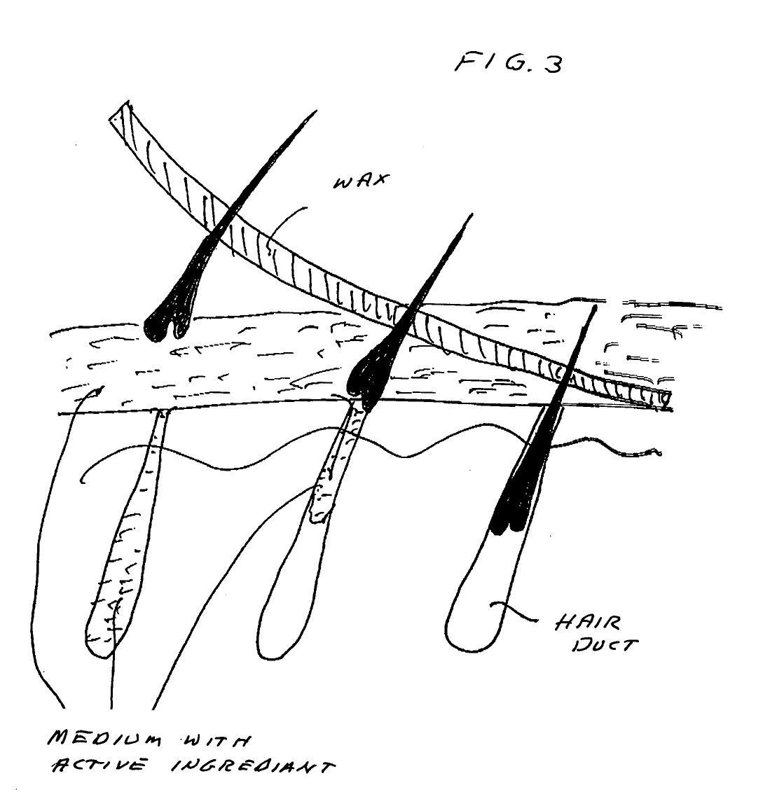

In another technique the skin region is immersed in the solution containing the stem cells and existing hair is pulled out of the skin. The stem cell containing solution is drawn into the hair duct as the hair is pulled out. FIGS. 2A, 2B and 3 show a preferred technique for inserting the stem cells into the hair ducts. The first step of the procedure is to wash a section of the skin to be treated with methyl alcohol and allowed to dry. A section of skin with growing hairs is depicted in FIG. 2A. Next step is to apply a liquid wax to the surface of the skin with a spatula, cover with a waxing paper stripe. Allow to wax to dry and a paper to adhere to the wax and hairs. Immerse skin into the medium containing stem cells and hair growth stimulation composition and pull out the paper stripe with the wax and hairs. The important step in this embodiment is to physically remove the hair shafts from the hair ducts in the skin section to be treated with the skin surface covered with stem cells to be delivered in the liquid medium. Applicant prefers using a commercially available wax marketed by Slect Spa Source of Sausilito, Calif. under the trade name Nature's Own Pine Wax although a wide variety of such waxes are available and would be satisfactory.

When hairs are in the process of pulling out from the hair ducts the negative pressure is created inside the duct. At the moment when hair bulb is leaving a hair duct infundibulum the surrounding fluid containing stem cells in cell medium hair removal rushes into empty hair canals filling it from the top to the bottom as shown in FIGS. 2B and 2C.

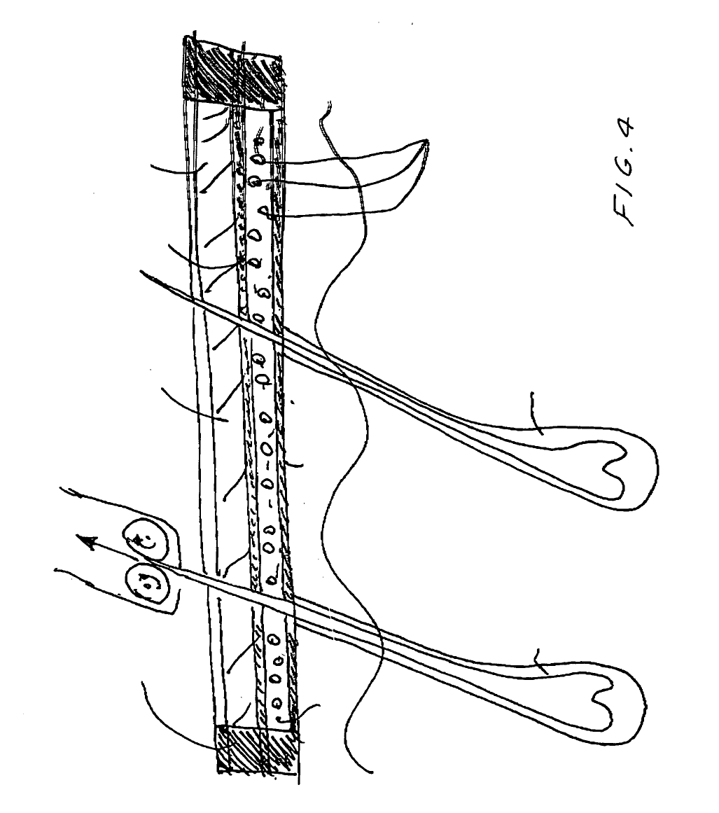

Hairs can be withdrawn one-at-a-time with tweezers. FIG. 4 shows a device for hair pulling with canister containing vaccine or encapsulated vaccine, melting from the body temperature membrane, covering cup with separating membrane.

### ILLUSTRATED EXAMPLE

The method of the invention is illustrated in the following example:
1. Stem cells were collected by punch biopsy from 102 healthy hair root canal bulge areas of an individual to be treated, and the samples were micro-surgically dissected to separate out and collect the undifferentiated stem cells from the mid-derm bulge area of hair papilla.
2. The collected stem cells were placed for cloning into Dulbecco's modified Eagle's medium (DMEM) with fetal calf serum as a culture medium.
3. When cells had proliferated and differentiated (approximately 21-28 days per one cycle) to about 40 cells per 1 cm³, the healthiest were selected and separated into three groups.
4. One group was frozen to −70° C. to create a bank of auto stem cells for fast reproduction when required. The second group was cloned in order for the secondary population to reach the cumulative population doublings (CDP) required, usually 2 to 10 times.
5. The third group was used for the preparation of a sterile suspension of stem cells in a carrier medium. The suspension was inoculated inter-dermally by pipette into recipient sites prepared on the scalp of the donor individual. Alternatively, the suspension was applied topically to the area being treated for hair re-growth, along with polypeptides expressed into the media by the stem cells during the cell culture mitotic process.
6. The areas inoculated with hair stem cells experienced increased hair growth and hair re-growth after about 21 to 28 days.

The method of hair growth via cell transplant of this invention provides the advantage that cloned stem cells can be expanded in culture so that the amount of donor material to be transplanted is not limited by the number of cells that can be harvested. Thus an individual with relatively few donor sites can provide enough stem cells to stimulate hair growth in a large area of skin, if so desired. In addition, the cloned cells can be implanted into the recipient sites without making more than superficial surgical incisions in the recipient sites. In contrast, many prior art hair grafting procedures require use of more extensive surgical techniques to implant the donor tissue.

### Other Variations

In an alternative embodiment, the solution delivered to the recipient site additionally contains polypeptides that trigger initiation of angiogenesis and neurogenesis, which are expressed into the media by the stem cells during the cell culture mitotic process. If desired, a portion of the cloned stem cells can be frozen and reserved for future inoculation into the individual undergoing hair growth treatment. If frozen to a temperature of about −70° C., a bank of auto stem cells can be kept for several months, allowing for fast expansion in culture when required.

The technique used to insert stem cells into hair ducts can also be used to insert other things. For example, the surrounding fluid may contain but not limited to the adipose, adult, hematopoietic stem cells, other hair growth stimulating compositions, proteins, enzymes, DNA, plasmids, vectors, micro-devices like extremely small antennas.

While the present invention has been described in terms of preferred embodiments, persons skilled in the art will recognize that many changes and modifications could be made. It is also possible to affect stem cell delivery to the skin for production of hair. Several factors are available for encouraging these effects. These factors include medication and nutritional supplements taken internally and the use of external factors such as ultrasound, laser light and microwave radiation. Stem cells may be mobilized into the skin region and blood flow by electromagnetic energy application, by an injection of medical solution or composition, by consuming nutritional product, by topical composition application and by mechanical skin region wounding or irritation. Holes in the skin may be made by sharp object to create a root canal for stem cells deposition, by light to create a root canal for stem cells deposition and by electromagnetic energy to create a root canal for stem cells deposition. Stem cell differentiation (sometime called plasticity) can be enhanced by medication, by nutritional supplement, by external electromagnetic energy and by external mechanical device, by topical compound application. Therefore, the scope of the invention is to be determined by the appended claims and their legal equivalents.

What is claimed is:

1. A method utilizing stem cells to stimulate hair growth on a patient comprising the steps of:
A) removing a number of living stems cells from the patient,
B) separating a number of stem cells from the removed tissue,
C) culturing the cells to either assure that they are differentiated into hair cells,
D) insert at least some number of stem cells into a skin region where hair growth is desired.

2. The method of claim 1 wherein the living stem cells are increase by cloning by a factor of at least 2

3. The method of claim 1 wherein the number of stem cells are increased by cloning by a factor of at least 10.

4. The method of claim 1 wherein the number of stem cells are increased by cloning by a factor of at least 1,000

5. The method of claim 1 wherein the number of stem cells are increased by cloning by a factor of at least 1,000,000

6. The method of claim 1 wherein the number of stem cells are transfected with cytokines.

7. The method of claim 1 wherein the number of stem cells are transfected with growth factors.

8. The method of claim 1 wherein the number of stem cells are transfected with genetic material.

9. The method of claim 1 wherein the stem cells are inserted into the skin region by scraping the skin region and topically applying a solution containing the stem cells to the region.

10. The method of claim 1 wherein the stem cells are inserted into the skin region by inoculation of the skin region with a stem cell containing solution.

11. The method of claim 1 wherein the stem cells are inserted into the skin region by immersing the skin region in a stem cell containing solution and pulling hairs out of the skin region allowing the solution to enter hair ducts.

12. The method of claim 1 wherein the stem cells are inserted into the skin region by injection into the skin a suspension of stem cell.

13. The method of claim 1 wherein the stem cells are mobilized into the skin region and blood flow by electromagnetic energy application.

14. The method of claim 1 wherein the stem cells are mobilized into the skin region and blood flow by injection of medical solution or composition.

15. The method of claim 1 wherein the stem cells are mobilized into the skin region and blood flow by consuming nutritional product.

16. The method of claim 1 wherein the stem cells are mobilized into the skin region and blood flow by topical composition application.

17. The method of claim 1 wherein the stem cells are mobilized into the skin region and blood flow by mechanical skin region wounding or irritation.

18. The method of claim 12 wherein the hairs are pulled out with tweezers.

19. The method of claim 12 wherein the hairs are pulled out using a waxing technique.

20. The method of claim 12 wherein the holes are made in the skin by sharp object to create a root canal for stem cells deposition.

21. The method of claim 12 wherein the holes are made in the skin by light to create a root canal for stem cells deposition.

22. The method of claim 12 wherein the holes are made in the skin by electromagnetic energy to create a root canal for stem cells deposition.

23. The method of claim 1 wherein the inserted to the skin stem cells differentiation are enhanced by medication.

24. The method of claim 1 wherein the inserted to the skin stem cells differentiation are enhanced by nutritional supplement.

25. The method of claim 1 wherein the inserted to the skin stem cells differentiation are enhanced by external electromagnetic energy.

26. The method of claim 1 wherein the inserted to the skin stem cells differentiation are enhanced by external mechanical device.

27. The method of claim 1 wherein the inserted to the skin stem cells differentiation are enhanced by topical compound application.
